SEQUENCE LISTING (15 sequences)

SEQ ID NO: 1 - protein, 130 aa, Artificial Sequence
  cAb-CEA3
QVQLVESGGGSVQPGGSLRLSCAASGSTGRGYYMGLFRQAPGKEREGVAAVWSGGGSTYYADSVQGRFTASQGNAKNIVYLQMNSLKPEDTALYYCAARTRREYNGRWYGPLDPRTYDYWGRGTQVTVSS

SEQ ID NO: 2 - DNA, 390 bp, Artificial Sequence
  cAb-CEA3
caggtgcagctggtggagtctgggggaggctcggtgcaacctggggggtctctgagactctcctgtgcagcctctggatcgactggcaggggctactacatgggcttgttccgtcaggctccagggaaggagcgcgagggggtcgcagctgtttggtctggtggtggtagcacatactatgccgactccgtgcagggccgattcaccgcctcccaaggcaacgccaagaatatagtgtatctgcaaatgaacagcctgaaacctgaggacactgccttgtactactgtgcagcacgtacccggcgcgagtacaatggtcgctggtacggccctctcgaccctcggacgtatgattactggggccgggggacccaggtcaccgtctcctca

SEQ ID NO: 3 - protein, 123 aa, Artificial Sequence [sequence omitted]

SEQ ID NO: 4 - DNA, 369 bp, Artificial Sequence
  cAb-CEA5
caggtgcagctggtggagtctgggggaggctcggtgcaggctggagggtctctgagactctcctgtgcggcctctggagacacctacggtagctactggatgggctggttccgccaggctccagggaaggagcgtgagggggtcgcagctattaataggggtggtggctatacagtctacgccgactccgtgaagggccgattcaccatctcccgagacaccgccaagaacacggtgtatctgcaaatgaacagcctgagacctgacgacacggccgactattactgtgcggctagcggggtactaggtggtttacatgaggactggtttaactactggggccaggggacccaggtcaccgtctcctca

SEQ ID NO: 5 - protein, 130 aa, Artificial Sequence
  cAb-CEA61
DVQLVESGGGSVQAGGSLRLSCAPSGYNIWTNSCMGWFRQAPGKEREGVALIYSGGGTTYYADSVKGRFTISQDNARNTVYLQMDSLKPEDTAMYYCAARRCGTYSNDLDVRTWNRYGFWGQGTQVTVSS

SEQ ID NO: 6 - DNA, 390 bp, Artificial Sequence
  cAb-CEA61
gatgtgcagctggtggagtctgggggaggctcggtgcaggctggagggtctctgaggctctcctgtgcgccctctggatacaacatctggactaacagctgcatgggctggttccgccaggctccagggaaggagcgcgagggggtcgcgttgatttattctggtggtggtacgacatactatgccgactccgtgaagggccgattcaccatctcccaagacaacgccaggaacacggtgtatctacaaatggacagcctgaaacctgaggacactgccatgtactactgtgccgcaaggaggtgtgggacctactcgaacgaccttgacgtccgaacttggaatcggtatggcttctggggccaggggacccaggtcaccgtctcctca

SEQ ID NO: 7 - protein, 117 aa, Artificial Sequence
  cAb-CEA72
QVQLVESGGGLVQPGGSLRLSCAASEFTFSSSYMSWVRQAPGKGLEWVSGINTDGSFTRYADSVKGRFTISRDNAKNTLYLQMNSLKSEDTALYYCAVGGGLGYGPRGQGTQVTVSS

SEQ ID NO: 8 - DNA, 351 bp, Artificial Sequence
  cAb-CEA72
caggtgcagctggtggagtctgggggaggcttggtgcaacctggggggtctctgagactctcctgtgcagcctctgaattcaccttcagtagcagctacatgagctgggtccgccaggctccagggaaggggctggagtgggtgtccggcattaataccgatggaagtttcacgcgctatgcagactccgtgaagggccgattcaccatctccagagacaacgccaagaacacgctgtatctgcaaatgaacagcctgaaatctgaggacacggccctgtattactgtgccgtaggcggcgggttaggctatggccccaggggccaggggacccaggtcaccgtctcctca

SEQ ID NO: 9 - protein, 15 aa, Artificial Sequence
  the llama gamma2c hinge region
AHHSEDPSSKAPKAP

SEQ ID NO: 10 - DNA, 33 bp, Artificial Sequence
  Fw primer
catgccatgactcgcggcccagccggccatggc

SEQ ID NO: 11 - DNA, 74 bp, Artificial Sequence
  Rev primer includes gamma2c hinge coding sequence
catgccatgggagctttgggagctttggagctggggtcttcgctgtggtgcgctgaggagacggtgacctgggt

SEQ ID NO: 12 - DNA, 30 bp, Artificial Sequence
  Fw primer
catgccatgggcacgccagtgtcagaaaaa

SEQ ID NO: 13 - DNA, 46 bp, Artificial Sequence
  Rev primer includes 6x his tag coding sequence
cgcgaattcttaatgatgatgatgatgatgctgtagcgcctggagg

SEQ ID NO: 14 - protein, 507 aa, Artificial Sequence
  immunoconjugate cAb-CEA5-beta-lactamase
  MISC_FEATURE (1)..(123): cAb-CEA5
  MISC_FEATURE (124)..(138): llama gamma2c hinge
  MISC_FEATURE (140)..(491): beta-lactamase
  MISC_FEATURE (492)..(497): 6xhis tag
QVQLVESGGGSVQAGGSLRLSCAASGDTYGSYWMGWFRQAPGKEREGVAAINRGGGYTVYADSVKGRFTISRDTAKNTVYLQMNSLRPDDTADYYCAASGVLGGLHEDWFNYWGQGTQVTVSSAHHSEDPSSKAPKAPMGTPVSEKQLAEVVANTITPLMKAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVEGSDSKVALAPLPVAEVNPPAPPVKASWVHKTGSTGGFGSYVAFIPEKQIGIVMLANTSYPNPARVEAAYHILEALQHHHHHH

SEQ ID NO: 15 - DNA, 1521 bp, Artificial Sequence
  cAb-CEA5-beta-lactamase
  misc_feature (1)..(369): cAb-CEA5
  misc_feature (370)..(415): llama gamma2c hinge
  misc_feature (422)..(1503): beta-lactamase
  misc_feature (1504)..(1521): 6xhis tag
caggtgcagctggtggagtctgggggaggctcggtgcaggctggagggtctctgagactctcctgtgcggcctctggagacacctacggtagctactggatgggctggttccgccaggctccagggaaggagcgtgagggggtcgcagctattaataggggtggtggctatacagtctacgccgactccgtgaagggccgattcaccatctcccgagacaccgccaagaacacggtgtatctgcaaatgaacagcctgagacctgacgacacggccgactattactgtgcggctagcggggtactaggtggtttacatgaggactggtttaactactggggccaggggacccaggtcaccgtctcctcagcgcaccacagcgaagaccccagctccaaagctcccaaagctccaatgggcacgccagtgtcagaaaaacagctggcggaggtggtcgcgaatacgattaccccgctgatgaaagcccagtctgttccaggcatggcggtggccgttatttatcagggaaaaccgcactattacacatttggcaaggccgatatcgcggcgaataaacccgttacgcctcagaccctgttcgagctgggttctataagtaaaaccttcaccggcgttttaggtggggatgccattgctcgcggtgaaatttcgctggacgatgcggtgaccagatactggccacagctgacgggcaagcagtggcagggtattcgtatgctggatctcgccacctacaccgctggcggcctgccgctacaggtaccggatgaggtcacggataacgcctccctgctgcgcttttatcaaaactggcagccgcagtggaagcctggcacaacgcgtctttacgccaacgccagcatcggtctttttggtgcgctggcggtcaaaccttctggcatgccctatgagcaggccatgacgacgcgggtccttaagccgctcaagctggaccatacctggattaacgtgccgaaagcggaagaggcgcattacgcctggggctatcgtgacggtaaagcggtgcgcgtttcgccgggtatgctggatgcacaagcctatggcgtgaaaaccaacgtgcaggatatggcgaactgggtcatggcaaacatggcgccggagaacgttgctgatgcctcacttaagcagggcatcgcgctggcgcagtcgcgctactggcgtatcgggtcaatgtatcagggtctgggctgggagatgctcaactggcccgtggaggccaacacggtggtcgagggcagcgacagtaaggtagcactggcgccgttgcccgtggcagaagtgaatccaccggctcccccggtcaaagcgtcctgggtccataaaacgggctctactggcgggtttggcagctacgtggcctttattcctgaaaagcagatcggtattgtgatgctcgcgaatacaagctatccgaacccggcacgcgttgaggcggcataccatatcctcgaggcgctacagcatcatcatcatcatcat

(19) United States
(12) Patent Application Publication
(10) Pub. No.: US 2007/0031430 A1
(43) Pub. Date: Feb. 8, 2007

(54) Immunoconjugates

(75) Inventors: Hilde Revets, Meise (BE); Virna Cortez-Retamozo, Brussel (BE); Serge Muyldermans, Hoelaart (BE); Patrick De Baetselier, Berchem (BE)

Correspondence Address: WOLF GREENFIELD & SACKS, PC, FEDERAL RESERVE PLAZA, 600 ATLANTIC AVENUE, BOSTON, MA, 02210-2206, US

(73) Assignees: Vlaams Interuniversitair Instituut, Zwijnaarde (BE); Vrije Universiteit Brussel, Brussel (BE)

(21) Appl. No.: 11/542,714

(22) Filed: Oct. 4, 2006

Related U.S. Application Data
(63) Continuation of application No. 10/885,492, filed on Jul. 6, 2004, now abandoned.
Application No. 10/885,492 is a continuation of application No. PCT/EP02/14842, filed on Dec. 23, 2002.

(30) Foreign Application Priority Data
Jan. 3, 2002 (EP) 02075048.5
Jul. 9, 2002 (EP) 02077734.8

(51) Int. Cl. A61K 39/395; C12N 9/00; C07K 16/30

(52) U.S. Cl. 424/155.1; 530/388.8; 435/188.5

(57) ABSTRACT

The present invention relates to novel immunoconjugates that are devoid of light chains and comprise at least one variable domain of a heavy chain antibody. The immunoconjugates of the present invention can be used for the preparation of a medicament to treat tumours.

### RELATED APPLICATIONS

This application is a continuation of U.S. application Ser. No. 10/885,492, filed Jul. 6, 2004, which is a continuation of PCT Application No. PCT/EP02/14842, filed Dec. 23, 2002, designating the United States of America, all of which are incorporated by reference herein.

### FIELD OF THE INVENTION

The present invention relates generally to biotechnology, and, more specifically, to novel immunoconjugates devoid of light chains that comprise at least one variable domain of a heavy chain antibody. The immunoconjugates described herein can be used, for example, for the preparation of a medicament to treat tumors.

### BACKGROUND OF THE INVENTION

The selective delivery of cytotoxic agents to tumour cells is desirable because systemic administration of these agents often kills normal cells within the body as well as the tumour cells sought to be eliminated. Targeted drug delivery systems provide a mechanism for delivering cytotoxic agents directly to cancerous cells.

Anti-tumor drug delivery systems currently in use typically utilize a cytotoxic agent conjugated to a tumor-specific antibody to form an immunoconjugate. This immunoconjugate binds to tumor cells and thereby “delivers” the cytotoxic agent to the site of the tumor. Basic research in the area of antibody-based tumor-targeted therapy has been driven for many years by the prospect of identifying surface antigens with sufficient restrictive tissue expression patterns to allow for the selective and specific accumulation of antibody in tumor tissue. the immunoconjugates utilized in these targeting systems including antibody-drug conjugates and antibody-toxin conjugates. Both polyclonal antibodies and monoclonal antibodies have been utilized in these immunoconjugates. Drugs used in these immunoconjugates include daunomycin, methotrexate, mitomycin C and vindesine. Toxins used in the antibody-toxin conjugates include bacterial toxins such as ricin and Pseudomonas aeruginosa exotoxin A. Despite the amount of research directed towards the use of immunoconjugates for therapeutic purposes, several limitations involved in these delivery approaches have become apparent. For example, the large amount of drug required to be delivered to the target tumor cell to effect killing of the cell is often unattainable because of limitations imposed by the number of tumor-associated antigens on the surface of the cells and the number of drug molecules that can be attached to any given antibody molecule.

This limitation has led to the use of more potent cytotoxic agents such as plant toxins in these conjugates and to the development of polymer-bound antibody-drug conjugates having very high drug multiplicity ratios. However, even with the large drug-loading ratios or with the use of potent toxins, many immunoconjugates still display suboptimal cytotoxic activity and are unable to effect complete killing at doses where all available antigenic sites are saturated. It has also been recognized that the cytotoxic activity of an immunoconjugate is often dependent on its uptake, mediated by the antibody component of the conjugate into the tumor cell. This internalization is crucial when using an antibody-drug conjugate in which the drug has an intracellular site of action or when using antibody-toxin conjugates. However, the vast majority of tumor-associated antigens and, thus, the antibody-drug or antibody-toxin conjugates bound to those antigens, are not internalized. Those conjugates that are internalized are often transported to the lysosome of the cell where the drug or toxin is degraded.

Accordingly, although an antibody-drug or antibody toxin conjugate may have excellent tumor-binding characteristics, the conjugate may nonetheless have a limited cytotoxic utility due to an inability to reach its site of action within the cell. Due to these drawbacks, the currently utilized anti-tumor drug or toxin delivery systems have had a limited amount of success, especially when used for in vivo treatment. Clinical trials have also demonstrated important limitations of mostly murine antibodies due to high immunogenicity, distribution to normal organs and poor penetration of solid tumors.

Along with the recent progress in genetic engineering techniques, there have been major efforts to construct or engineer antibodies to obtain smaller binding units that retained the specificity and affinity of classical antibodies and/or to reduce the immunogenicity of the murine molecules (“humanization”) (Hudson, 1998). The variable fragment (Fv) composed of the paired variable domain of the immunoglobulin heavy chain (VH) and the variable domain of the immunoglobulin light chain (VL) is the smallest, intact antigen-binding fragment one can obtain from a conventional antibody. However, it is more convenient to produce Fv as recombinant single-chain Fv (scFv), i.e., an Fv where the VH and VL domains are tethered by a flexible oligopeptide linker (Bird et al., 1988). To broaden the immunotherapeutic potential, more complex constructs have been engineered, e.g., by linking two different scFvs to bridge tumor cells with either T or NK cells (bispecific antibodies) or a scFv attached to a toxin or an enzyme to act on a pro-drug (Hudson, 1999). However, several of these scFv-based constructs proved difficult to express and purify, and exhibited several serious shortcomings in functionality. Common hurdles were the tendency to form aggregates due to the presence of an oligopeptide linker, the susceptibility of the linker to proteolytic cleavage and subsequent unfolding of the antibody constructs (Whitlow et al., 1993).

The naturally occurring heavy-chain antibodies devoid of light chain and of CH1 domain that were discovered in camelids (Hamers-Casterman et al., 1993) may constitute a promising alternative in this respect but they have never evaluated as immunoconjugates. The observation that camelids possess large amounts of functional heavy-chain antibodies lacking light chains formed the basis for generating functional single-domain antibody fragments (referred to as cAb for camel single-domain antibody) (Ghahroudi et al., 1997; Lauwereys et al., 1998) from their variable domains (V_{H}H). These small-sized molecules are well expressed and were shown to overcome to a large extent the solubility, aggregation and degradation problems often encountered with scFvs. Furthermore, they show good specificity towards their corresponding antigens and can be obtained with affinities comparable to scFvs (Muyldermans and Lauwereys, 1999; Riechmann and Muyldermans, 1999). However, due to the number of complex parameters involved (efficiency of tumor targeting, efficiency of internalization, efficiency of killing tumors, immunogenicity, problems of expression) it cannot be predicted whether a particular class of immunoconjugate will be successful or not.

### DISCLOSURE OF THE INVENTION

We have constructed immunoconjugates which are fusions between camelid variable heavy chain antibodies and an enzyme and have surprisingly found that these immunoconjugates have superior in vivo characteristics such as lower immunogenicity and a superior killing of tumor cells in comparison to existing immunoconjugates.

Disclosed are immunoconjugates that comprise a fusion between at least one variable domain of a heavy chain antibody and an anti-tumor agent. It is understood that a particular immunoconjugate has a specificity for at least one tumor antigen. Various tumor antigens or tumor markers are known in the art and it has been proposed that therapy against tumors expressing these markers can be achieved by using specific immunoconjugates. The word “tumor” is to be understood as referring to all forms of neoplastic cell growth including carcinomas, sarcomas, lymphomas and leukemias. Thus, such an immunoconjugate comprises a variable domain of a heavy chain antibody that has been linked to a anti-tumor agent. An “anti-tumor agent” is understood to be a cytotoxic agent (e.g., a toxin) or an enzyme capable of converting a pro-drug into an active cytotoxic agent.

As described herein, the immunoconjugate is devoid of any light chain, but includes at least one heavy chain antibody. Preferably, the variable domain of a heavy chain antibody is derived from camelids, but it can also be derived from other species (e.g., mouse, human). The variable domain of a heavy chain antibody has an anti-tumor agent attached to it. It is desirable that the antibody have a good affinity for its tumor marker (its target). This is so that once the antibody has reached its target, it remains bound to that target for a sufficient amount of time to achieve the desired result, for example, cytotoxicity. In addition, the antibody should have good specificity for the target antigen so that binding to non-target antigens does not occur to any significant degree.

Thus, in a first embodiment, the invention provides an immunoconjugate, devoid of a light chain, specifically binding to a tumor antigen comprising at least one single-domain variable domain of a heavy chain antibody having an anti-tumor agent attached thereto and further characterized by inhibiting the growth of tumor cells expressing the tumor antigen and leads to a reduction in tumor mass. The wording “inhibiting the growth” comprises shrinking the tumor, inducing necrotic lesions in the tumor, inducing tumor death and paralyzing the growth of a tumor. In a preferred embodiment, the reduction in tumor mass is at least 50%, 60%, 70%, 80% and, preferentially, more than 90%.

The conjugation (or coupling) between the single-domain variable heavy chain antibody and, for example, a prodrug converting enzyme or a toxin can be effected by chemical bonding or by splicing together nucleic acid sequences that code for both partners.

In a particular embodiment, the immunoconjugate is bivalent and formed by bonding, chemically or by recombinant DNA techniques, together two monovalent variable domain of heavy chains. The immunoconjugate can also be bispecific and formed by bonding together two variable domains of heavy chains, each one specific for a different tumor marker.

In another embodiment, the invention provides an immunoconjugate, devoid of a light chain, specifically binding to carcinoembryonic antigen (“CEA”), but comprising at least one variable domain of a heavy chain antibody having an anti-tumor agent attached thereto and further characterized by inhibiting the growth of tumor cells expressing CEA.

CEA has been used as a marker antigen for cancer imaging and therapy. A large number of CEA antibodies with different specificities and affinities are known in the art. An optimal anti-CEA antibody is an antibody that has a higher proportion and amount of the antibody localized to tumor rather than to other body tissues and it is said that it is “specifically binding to.” Preferably, no non-specific antibody localization is observed. The specificity of an anti-CEA immunoconjugate is preferably such that it binds to human colorectal carcinoma but does not bind to some or all of the following normal tissues: liver, kidney, large intestine, tonsil, lung, brain, testis, ovary, cervix, breast, blood films, placenta, spleen, thyroid, esophagus, stomach, pancreas, lymph node, and skeletal muscle.

An immunoconjugate according to the invention includes at least one variable domain of a heavy chain antibody that is linked to an anti-tumor agent. This allows the antibody to target the anti-tumor agent to the tumor and hence results in inhibition of growth but preferably damage, destruction and/or killing of the tumor. Thus, the immunoconjugate is suitable for use in a method of treatment of the human or animal body. In particular, the immunoconjugate with a specificity for CEA is suitable for use in the manufacture of a medicament to treat a colorectal tumor. The anti-tumor agent linked to the antibody may be any agent that inhibits, destroys, damages or kills a tumor to which the antibody has bound or in the environment of the cell to which the antibody has bound. For example, the anti-tumor agent may be a toxic agent such as a chemotherapeutic agent, a radioisotope, an enzyme which activates a prodrug or a cytokine. Suitable chemotherapeutic agents are known to those skilled in the art and include anthracyclines (e.g., daunomycin and doxorubicin), methotrexate, vindesine, neocarzinostatin, cis-platinum, chlorambucil, cytosine arabinoside, 5-fluorouridine, melphalan, ricin and calicheamicin. The chemotherapeutic agents may be conjugated to the antibody using conventional methods known in the art. Suitable radioisotopes for use as anti-tumor agents are also known to those skilled in the art. For example ¹³¹I or astatine such as ²¹¹ At may be used. These isotopes may be attached to the antibody using conventional techniques known in the art. The anti-tumor agent which is attached to the antibody may also be an enzyme which activates a prodrug. This allows activation of an inactive prodrug to its active, cytotoxic form at the tumor site as is undertaken in the so-called “antibody-directed enzyme prodrug therapy” (ADEPT). In clinical practice, the antibody-enzyme conjugate is administered to the patient and allowed to localize in the region of the tumor to be treated. The prodrug is then administered to the patient so that conversion to the cytotoxic drug is localized in the region of the tumor to be treated under the influence of the localized enzyme. One enzyme is bacterial carboxypeptidase G2 (CPG2) whose use is described in, for example, PCT International Patent Publication No. WO 88/07378. Another bacterial enzyme is beta-lactamase whose use is described in U.S. Pat. No. 5,773,435. The antibody-enzyme conjugate may be modified in accordance with the teaching of PCT International Patent Publication No. WO 89/00427, in order to accelerate clearance from areas of the body not in the vicinity of a tumor. The antibody-enzyme conjugate may also be used in accordance with PCT International Patent Publication No. WO 89/00427 by providing an additional component which inactivates the enzyme in areas of the body not in the vicinity of the tumor. The anti-tumor agent conjugated to the antibody may also be a cytokine such as interleukin-2 (IL-2), interleukin-12 (IL-12), granulocyte-macrophage colony-stimulating factor (GM-CSF) or tumor necrosis factor alpha (TNF-alpha). The antibody targets the cytokine to the tumor so that the cytokine mediates damage to or destruction of the tumor without affecting other tissues. The cytokine may be fused to the antibody at the DNA level using conventional recombinant DNA techniques.

In another embodiment, the invention provides an immunoconjugate, devoid of a light chain, specifically binding to a tumor antigen, but comprising at least one variable domain of a heavy chain antibody derived from camelids having an anti-tumor agent attached thereto and further characterized by inhibiting the growth of tumor cells expressing the tumor antigen.

In the present invention, a variable domain of a heavy chain antibody derived from a camelid is designated as V_{H}H.

In another embodiment, the invention provides an immunoconjugate, devoid of a light chain, specifically binding to CEA, but comprising at least one variable domain of a heavy chain antibody, derived from camelids, having an anti-tumor agent attached thereto and further characterized by inhibiting the growth of tumor cells expressing CEA.

In the family of “camelids,” immunoglobulins devoid of light polypeptide chains are found. “Camelids” comprise old world camelids (Camelus bactrianus and Camelus dromaderius) and new world camelids (for example, Lama paccos, Lama glama and Lama vicugna). European Patent Office Publication EP0656946 (corresponding to U.S. Pat. No. 6,015,695 (Jan. 18, 2000) to Casterman et al.) describes the isolation and uses of camelid immunoglobulins and is incorporated herein by this reference.

In another embodiment, the invention provides an immunoconjugate, devoid of a light chain, specifically binding to a tumor antigen, but comprising at least one variable domain of a heavy chain antibody having an enzyme which activates a prodrug attached thereto and further characterized by inhibiting the growth of tumor cells expressing the tumor marker.

In a particular embodiment, the enzyme is bacterial beta-lactamase.

In a more particular embodiment, the immunoconjugate has the nucleotide sequence set forth in SEQ ID NO:15 (of the accompanying and incorporated SEQUENCE LISTING) and the amino acid sequence set forth in SEQ ID NO:14.

In another embodiment, the immunoconjugates described hereinbefore can be used as a medicament.

In another embodiment, the immunoconjugate provided by the invention can be used for the manufacture of a medicament to treat tumors expressing a tumor marker that is recognized by the immunoconjugate.

In yet another embodiment, the invention provides a pharmaceutical composition comprising an immunoconjugate of the present invention.

### BRIEF DESCRIPTION OF FIGURES

FIG. 1: Structures of the cephalosporin mustard prodrug CCM and the parent drug phenylene-diamine mustard PDM.

FIG. 2: Cytotoxic effects of cAb-CEA5-βL+CCM combinations on LS 174T adenocarcinoma cells as determined by the incorporation of [³H] thymidine into DNA. The LS 174T cells were incubated with the cAb-CEA5-βL conjugates, washed and treated with CCM for 1 h. The effects were compared to cells treated with CCM or PDM for 1 h without prior conjugate exposure and to cells that were treated with saturating amounts of unconjugated cAb-CEA5 prior to conjugate treatment.

FIG. 3: In vitro cytotoxicity of CCM (3 μM) on LS 174T adenocarcinoma cells. The cells were treated with varying concentrations of the conjugates, washed and then exposed to CCM for 1 h. After 24 h incubation and pulsing for 18 h, cytotoxicity was quantified by measuring [³H] thymidine incorporation relative to untreated control cells. Demonstration of the immunological specificity of prodrug activation was done by treating the cells with the non-binding control conjugate cAb-Lys3-βL prior to CCM exposure or by saturation with non-conjugated cAb-CEA5 (0.1 mg/ml) prior to conjugate treatment.

FIG. 4: Pharmacokinetics of cAb-CEA5::βL and the nonbinding control cAb-Lys3:: βL in nude mice (three animals/group). βL conjugate levels in subcutaneous LS174T colon carcinoma tumors and in normal tissues are shown at 6 h, 24 h and 48 h post administration. cAb-Lys3::βL served as nonbinding control.

FIG. 5: Therapeutic effect of cAb::βL/CCM combinations in nude mice with LS174T xenografts. Conjugates (1 mg/kg) were injected iv on days indicated by the arrows, and CCM was administered 24 h later. The therapeutic effects were compared to those of PDM at the MTD.

### DETAILED DESCRIPTION OF THE INVENTION

The term “medicament to treat” relates to a composition comprising immunoconjugates as described herein and a pharmaceutically acceptable carrier or excipient (both terms can be used interchangeably) to treat or to prevent diseases as described herein. The administration of an immunoconjugate as described herein or a pharmaceutically acceptable salt thereof may be by way of oral, inhaled or parenteral administration. The active compound may be administered alone or preferably formulated as a pharmaceutical composition. An amount effective to treat tumors that express the antigen recognized by the immunoconjugate depends on the usual factors such as the nature and severity of the disorders being treated and the weight of the mammal. However, a unit dose will normally be in the range of 0.01 to 50 mg, for example 0.01 to 10 mg, or 0.05 to 2 mg of immunoconjugate or a pharmaceutically acceptable salt thereof. Unit doses will normally be administered once or more than once a day, for example 2, 3, or 4 times a day, more usually 1 to 3 times a day, such that the total daily dose is normally in the range of 0.0001 to 1 mg/kg; thus a suitable total daily dose for a 70 kg adult is 0.01 to 50 mg, for example 0.01 to 10 mg or more usually 0.05 to 10 mg.

It is preferred that the compound or a pharmaceutically acceptable salt thereof be administered in the form of a unit-dose composition, such as a unit-dose oral, parenteral, or inhaled composition. Such compositions are prepared by admixture and are suitably adapted for oral, inhaled or parenteral administration and, as such, may be in the form of tablets, capsules, oral liquid preparations, powders, granules, lozenges, reconstitutable powders, injectable and infusable solutions or suspensions, suppositories or aerosols.

Tablets and capsules for oral administration are usually presented in a unit dose and contain conventional excipients such as binding agents, fillers, diluents, tabletting agents, lubricants, disintegrants, colorants, flavorings, and wetting agents. The tablets may be coated according to well known methods in the art. Suitable fillers for use include cellulose, mannitol, lactose and other similar agents. Suitable disintegrants include starch, polyvinylpyrrolidone and starch derivatives such as sodium starch glycolate. Suitable lubricants include, for example, magnesium stearate. Suitable pharmaceutically acceptable wetting agents include sodium lauryl sulphate. These solid oral compositions may be prepared by conventional methods of blending, filling, tabletting or the like. Repeated blending operations may be used to distribute the active agent throughout those compositions employing large quantities of fillers. Such operations are, of course, conventional in the art. Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example, sorbitol, syrup, methyl cellulose, gelatin, hydroxyethylcellulose, carboxymethyl cellulose, aluminum stearate gel or hydrogenated edible fats; emulsifying agents, for example, lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid; and, if desired, conventional flavoring or coloring agents. Oral formulations also include conventional sustained release formulations, such as tablets or granules having an enteric coating. Preferably, compositions for inhalation are presented for administration to the respiratory tract as a snuff or an aerosol or solution for a nebulizer, or as a microfine powder for insufflation, alone or in combination with an inert carrier such as lactose. In such a case, the particles of active compound suitably have diameters of less than 50 microns, preferably less than 10 microns, for example, between 1 and 5 microns, such as between 2 and 5 microns. A favored inhaled dose will be in the range of 0.05 to 2 mg, for example, 0.05 to 0.5 mg, 0.1 to 1 mg or 0.5 to 2 mg.

For parenteral administration, fluid unit dose forms are prepared containing a compound of the present invention and a sterile vehicle. The active compound, depending on the vehicle and the concentration, can be either suspended or dissolved. Parenteral solutions are normally prepared by dissolving the compound in a vehicle and filter sterilizing before filling into a suitable vial or ampoule and sealing. Advantageously, adjuvants such as a local anesthetic, preservatives and buffering agents are also dissolved in the vehicle. To enhance the stability, the composition can be frozen after filling into the vial and the water removed under vacuum. Parenteral suspensions are prepared in substantially the same manner except that the compound is suspended in the vehicle instead of being dissolved and sterilized by exposure to ethylene oxide before suspending in the sterile vehicle. Advantageously, a surfactant or wetting agent is included in the composition to facilitate uniform distribution of the active compound. Where appropriate, small amounts of bronchodilators, for example, sympathomimetic amines such as isoprenaline, isoetharine, salbutamol, phenylephrine and ephedrine; xanthine derivatives such as theophylline and aminophylline; corticosteroids such as prednisolone; and adrenal stimulants such as ACTH may be included. As is common practice, the compositions will usually be accompanied by written or printed directions for use in the medical treatment concerned.

The present invention further provides a pharmaceutical composition for use in the treatment and/or prophylaxis of the herein described disorders, which pharmaceutical composition comprises the immunoconjugate, a pharmaceutically acceptable salt thereof, or a pharmaceutically acceptable solvate thereof, and, if required, a pharmaceutically acceptable carrier thereof.

It should be clear that the therapeutic method of the present invention against tumors can also be used in combination with any other tumor therapy known in the art such as irradiation, chemotherapy or surgery.

The following examples more fully illustrate preferred features of the invention, but are not intended to limit the invention in any way. All of the starting materials and reagents disclosed below are known to those skilled in the art and are available commercially or can be prepared using well-known techniques.

### EXAMPLES

### 1. Construction and Purification of the Camel Single-Domain::β-lactamase Conjugates

Several anti-CEA camel single domain VH and V_{H}H antibodies were retrieved from an immunized phage display library. FACS analysis was performed to analyze the ability of these antibodies to recognize CEA expressed on LS 174T cells (the human LS 174T adenocarcinoma cell line was obtained from ATCC (Manassas, Va.). LS 174T is a trypsinized variant of the LS 180 colon-adenocarcinoma cell line and produces large amounts of carcinoembryonic antigen (CEA).

Based on the FACS profiles, V_{H}Hs cAb-CEA3 (SEQ ID NO: 1 for the amino acid sequence and SEQ ID NO: 2 for the nucleotide sequence), cAb-CEA5 (SEQ ID NO: 3 for the amino acid sequence and SEQ ID NO: 4 for the nucleotide sequence), cAb-CEA61 (SEQ ID NO: 5 for the amino acid sequence and SEQ ID NO: 6 for the nucleotide sequence) and the VH cAbCEA72 (SEQ ID NO: 7 for the amino acid sequence and SEQ ID NO: 8 for the nucleotide sequence) were chosen for conjugate construction. cAb-CEA-β-lactamase conjugates were constructed in a stepwise fashion by insertion of the cAb-CEA sequence, the llama γ2c hinge (AHHSEDPSSKAPKAP) region sequence (SEQ ID NO: 9) and the β-lactamase (bL) gene followed by a 6xhis-tag into the pHEN6 expression vector. The particular bL was cloned from the E. cloacae P99 strain by PCR amplification. Primer-sequences used are 5′-CATGCCATGACTCGCGGCCCAGCCGGCCATGGC-3′ (Fw primer) (SEQ ID NO: 10) and 5′-CATGCCATGGGAGCTTTGGGAGCTTTGGAGCT GGGGTCTTCGCTGTGGTGCGCTGAGGAGACGGTGACCTGGGT-3′ (Rev primer: includes γ2c hinge coding sequence) for amplification and NcoI cloning of cAb-CEA/γ2c hinge (SEQ ID NO: 11).

5′-CATGCCATGGGCACGCCAGTGTCAGAAAAA-3′ (Fw primer) (SEQ ID NO: 12) and 5′-CGCGAATTCTTAATGATGATGATGATGATGCTGTAGCGCCTGGAGG-3′ (Rev primer: includes 6x his tag coding sequence) for amplification and directional NcoI-EcoRI cloning of β-lactamase (SEQ ID NO: 13). The resulting cAb-CEA-βL his-tagged conjugates were expressed in E. coli and purified on an IMAC column (Ni-NTA Superflow, QIAGEN) followed by gel filtration on a Superdex 75 HR 10/30 column (Pharmacia). The anti-lysozyme camel single-domain antibody cAb-Lys3 conjugated to β-lactamase was also engineered and used as non-binding control in further experiments. The isolation of the cAb-Lys3 antibody fragment was previously described (Ghahroudi et al., 1997). The gene was recloned in an expression vector under control of the lac promoter, between the Pel B leader signal and a carboxyterminal hexahistidine tail (Lauwereys et al., EMBO J, 17, 3512-3520 (1998).

Enzymatic activity assays of the bL portion of the conjugates were undertaken using nitrocefin as the substrate. Michaelis-Menten kinetic analyses confirmed that the fusion protein retained the full enzymatic activity from the enzyme from which it was derived.

### 2. In vitro Cytotoxicity Assays Using cAb-CEA5-βL Conjugate

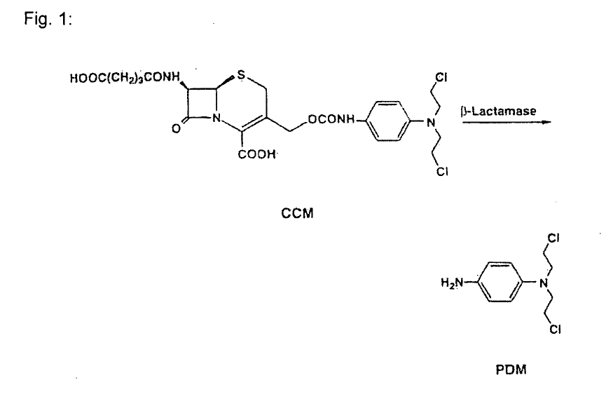

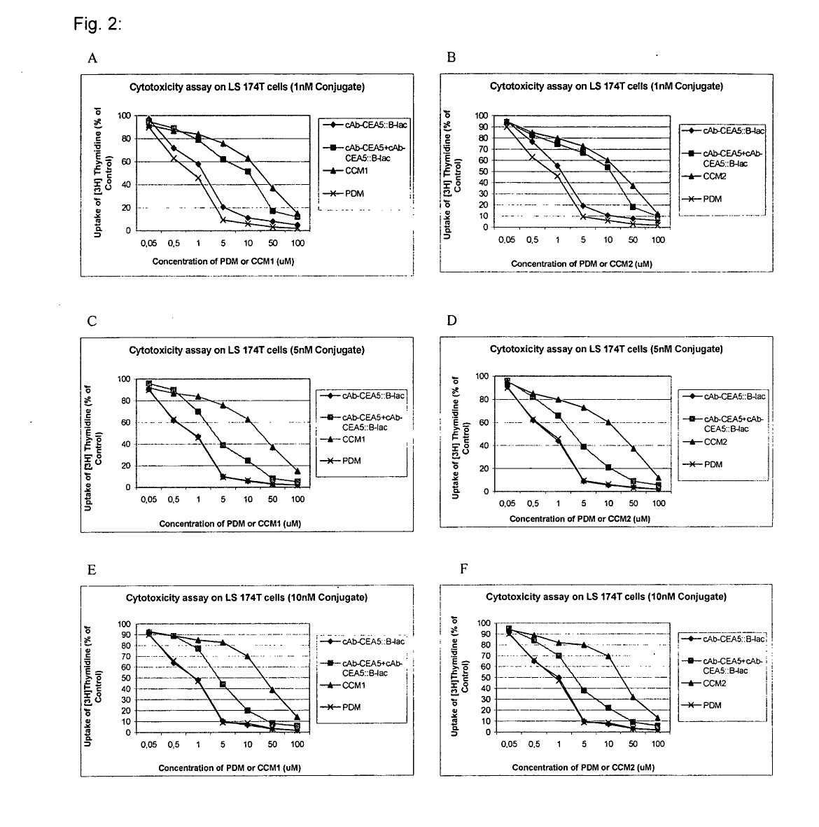

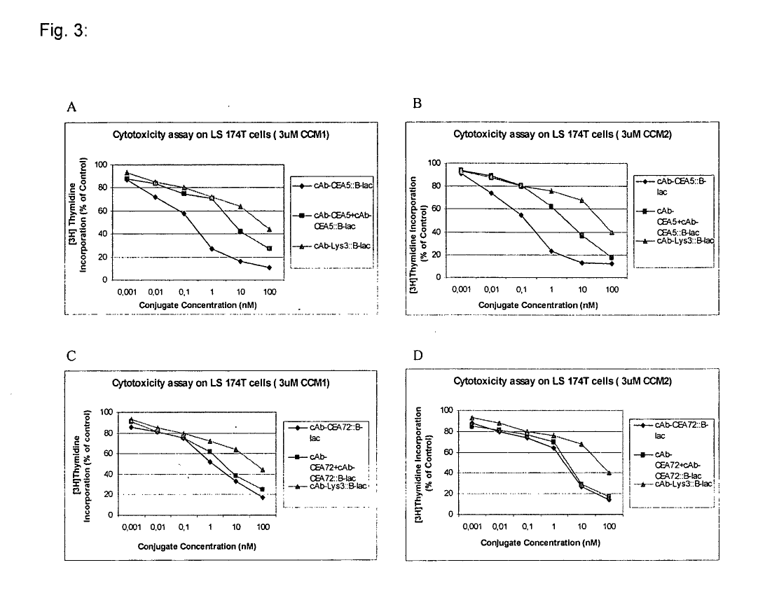

A total of 10⁴ LS 174T human adenocarcinoma cells/well (0.1 ml of EMEM with 10% fetal bovine serum, 100 units/ml penicillin, 0.1 mg/ml streptomycin, 1 mM sodium pyruvate and 0.1 mM non-essential amino acids) were plated into 96-well microtiter plates and allowed to adhere overnight. For blocking experiments, the cells were incubated with unconjugated cAb-CEA5 at 0.1 mg/ml for 30 minutes prior to treatment with the cAb-CEA-βL conjugates. The cells were then exposed to the conjugates at 1, 5, and 10 nM. After 30 minutes at 4° C., the plates were washed 3 times with antibiotic free RPMI 1640 medium with 10% fetal bovine serum, and then different amounts of the prodrug CCM (7-(4-carboxy-butanamido) cephalosporin mustard) or PDM (parental drug, phenylenediamine mustard) were added (see FIG. 1 for the structure). CCM and PDM were also added to cells that were not treated with the conjugates. We received the prodrug CCM and parental drug PDM for the in vitro cytotoxicity studies from Dr. Peter Senter (Director Chemistry, Seattle Genetics, Inc., Washington, U.S.A). After 1 hour at 37° C., the cells were washed with EMEM and incubated for 24 hours. The cells were then pulsed for 18 hours with [³H] thymidine (1 μCi/well) at 37° C., detached by freezing and thawing, and harvested onto glass fiber filter mats using a 96-well cell harvester. Radioactivity was counted using a β-plate counter. Another set of experiments was performed with varying concentrations of the anti-CEA-βL conjugates or cAb-Lys3-βL as a non-binding control. After conjugate exposure, cells were treated with a fixed amount of CCM. After 24 hours incubation, the cells were pulsed for 18 hours, harvested and radioactivity was counted with a β counter. The cytotoxic effects of a conjugated V_{H}H, cAb-CEA5-βL (SEQ ID NO: 14 for the amino acid sequence and SEQ ID NO: 15 for the nucleotide sequence) (FIG. 2) in combination with CCM prodrug were determined on LS 174T human adenocarcinoma cells which express the CEA antigen. The cells were exposed to the conjugate, washed to remove unbound material, and treated with various amounts of two different batches of CCM (CCM1, CCM2). Cytotoxic activity was determined by measuring the incorporation of [³H] thymidine into DNA relative to untreated cells. The prodrug CCM was approximately 40 fold less toxic to LS 174T cells than the parental drug PDM. cAb-CEA5-βL effectively activated the prodrug in a dose dependent manner, leading to a cytotoxicity equivalent in activity to PDM. Prodrug activation was immunologically specific since cAb-CEA5-βL activated CCM at marginal levels on cells that were saturated with unconjugated cAb-CEA5 prior exposure to the fusion protein. In addition, to compare the relative abilities of the cAb-CEA-βL conjugate for prodrug activation, LS 174T cells were exposed to various amounts of conjugate. Unbound material was washed off, and CCM was added at a fixed concentration of 3 μM, which has low cytotoxic activity in the absence of β-lactamase. cAb-CEA5-βL induced effectively the prodrug in a dose dependent manner and showed to be immunologically specific (FIG. 3 panel A and B). Demonstration of the immunological specificity of prodrug activation was done by saturation with non-conjugated cAb-CEA or by treating the cells with non- binding control conjugate, cAb-Lys3-βL, prior to CCM. As expected, cAb-Lys3-βL did not activate the prodrug CCM.

### 3. Immunogenicity Studies

To study the immune response to cAb-enzyme conjugates, BALB/c mice receive a single or multiple course of intravenous treatment with cAb-CEA5 antibody fragments conjugated to bacterial enzyme β-lactamase (1 mg of immunoconjugate/kg bodyweight). The development of mouse anti-camel antibodies and anti-β-lactamase antibodies is analyzed at day 7, 14 and 60 after the last treatment course by ELISA. Anti-β-lactamase antibodies present in serum of mice are tested for their capacity to inhibit β-lactamase activity in vitro.

### 4. In vivo Therapy Experiments in Nude Mice Bearing LS 174T Carcinoma Tumour Xenografts

4.1 Conjugate Localization

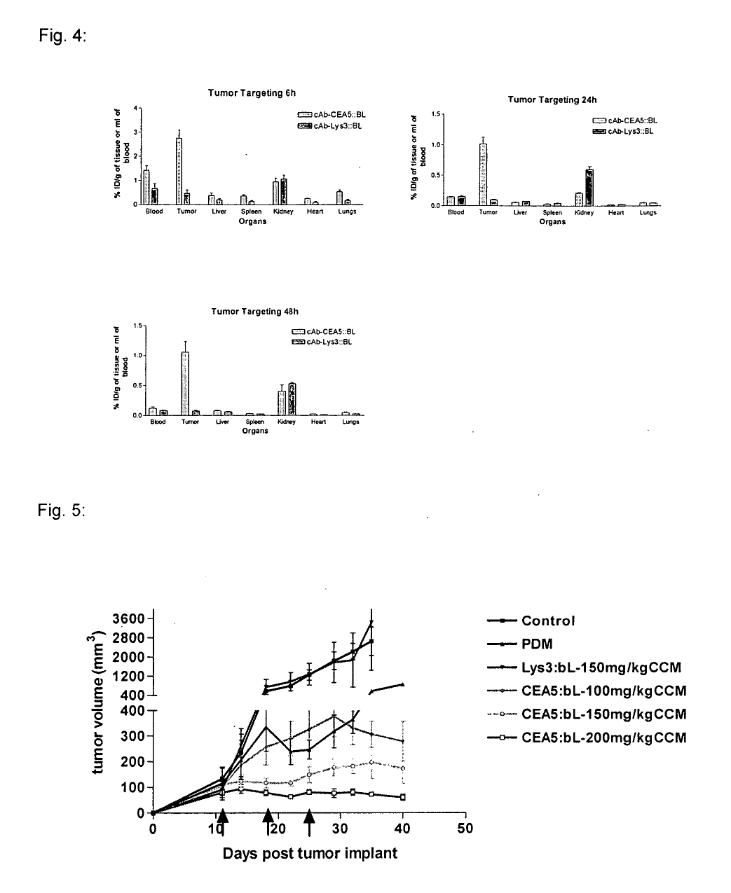

Studies were undertaken in nude mice to establish the extent of cAb-CEA5::β-lactamase conjugate localization in LS 174 T tumor xenografts. ¹²⁵I labeled cAb-CEA5::β-lactamase (4.728.481 cpm/μg conjugate) or cAb-Lys3::β-lactamase conjugate (2.691.621 cpm/μg conjugate) were injected i.v. (1 mg/kg) into mice (3 animals/group) that had subcutaneous LS 174T carcinoma tumors of about 0.5-1 cm diameter. The amount of radioactivity in the tumors, blood, and several other tissues was determined 6 h, 24 h and 48 h later (FIG. 4 ). It was found that the concentration of cAb-CEA5::βL in tumors was much higher (>10-fold) than in any other of the tissues measured. This was most likely due to binding to the CEA antigen on tumor cells, since the irrelevant cAb-Lys3::βL showed no preferential intratumoral accumulation. We also noticed a rather high accumulation of both cAb-CEA5::βL and cAb-Lys3::βL conjugates in the kidneys (0.41-0.53% ID/g tissue). In order to see whether the radioactivity measured originated from intact conjugate molecules or degraded material, we assessed the enzymatic activity of β-lactamase in targeted tumor, liver and kidney tissue using nitrocefin. The results showed that enzymatic activity was intact in the excised tumor tissue whereas no activity could be measured in liver nor kidney tissue, indicating that the radioactivity measured in kidney and liver tissue was not derived from intact antibody-enzyme conjugate molecules (spiking these tissue suspensions with similar concentrations of cold cAb-CEA5::βL resulted in positive enzymatic activity, indicating that the tissue suspensions did not exhibit inhibitory activity on the enzymatic activity). Maximal tumor uptake of approximately 3% injected dose/g tumor was seen 6 h after dosing of the cAb-CEA::βL conjugate whereas no targeting was seen for the nonbinding control cAb-Lys3::βL conjugate. The blood and normal tissue levels were still high at this time-point and thus tumor/normal tissue ratios were low. After 24 h, although the amounts of cAb-CEA::βL conjugate in the tumors had fallen to approx. 1% injected dose/g tumor, the blood and normal tissue levels had fallen more rapidly, and consequently, tumor/normal tissue ratios were in the 10-50 fold range, except for the kidneys were a high amount of radioactivity could still be measured. After 48 h, a similar biodistribution was seen. Based on these data, an interval of 24 h between conjugate and prodrug administration was selected for antitumour studies.

4.2 Antitumour Effect of the Prodrug Therapy

The antitumor effect of giving cAb-CEA::βL conjugate (1 mg/kg body weight) followed 24 h later by escalating doses of CCM (100, 150, 200 mg/kg) are shown in FIG. 5. The prodrug therapy combination gave a significant antitumor effect compared to non-treated tumor-bearing mice or mice receiving prodrug in combination with the nonbinding cAb-Lys3::βL conjugate. Therapeutic efficiency was dose-dependent. Significant antitumor activity including partial regression of the tumors was obtained in the animals that received 200 mg CCM/kg/injection. There were no apparent toxicities in any of the groups receiving CCM therapy. In contrast, systemic treatment of mice with the drug PDM at 4 mg/kg/injection had no beneficial effect on tumor growth since they grew out after the treatment was discontinued. Moreover, although the PDM dose given at about the maximal tolerated dose (4.5 mg/kg/injection), systemic administration led to toxicity and resulted in >10% body weight loss.

Materials and Methods

Tumor Localization Studies

The cAb-CEA::βL conjugate was radioiodinated with carrier-free ¹²⁵I using the IODOGEN reagent, following the manufacturer's (Pierce, Rockford, Ill., USA) recommended method. In vitro retention of immunoreactivity postradioiodination was confirmed by binding to LS174T cells. Approximately 1 mg of conjugate/kg body weight were injected intravenously into athymic nude mice bearing established tumor xenografts (2×10⁶ LS174T tumor cells injected 10 days previously and tumors measured approximately 5-6 mm in diameter). Following injection of the conjugate, groups of three mice were killed 6, 24 and 48 h later. The tumor, a sample of blood, and a range of other tissues were removed, weighed, and counted in a gamma counter.

Antitumor Studies

Groups of 5 female athymic nude mice were injected subcutaneously with 2×10⁶ LS174T tumor cells. Ten days later when the tumors reached a size of about 100 mm³, 1 mg/kg bodyweight of βL conjugates was injected iv, followed 24 h later by the prodrug CCM. Treatment with cAb-βL+CCM was carried out on a weekly schedule for a total of 3 rounds. The animals were monitored twice a week for general health, weight and tumor growth and compared to control groups receiving no treatment. Tumor volumes were calculated using the formula (longest length×perpendicular width²)÷2.

### REFERENCES

Adams, G. P., Schier, R., Marshall, K., Wolf, E. J., McCall, A. M., Marks, J. D. and Weiner, L. M., Increased affinity leads to improved selective tumour delivery of single-chain Fv antibodies. Cancer Res, 58, 485-490 (1998a).
Adams G P, Schier R, McCall A M, Crawford R S, Wolf E J, Weiner L M and Marks J D. Prolonged in vivo tumour retention of a human diabody targeting the extracellular domain of human HER-2/neu. Br. J. Cancer, 77,1405-1412 (1998b).
Adams, G. P., Schier R., McCall A. M., Simmons H. H., Horak E. M., Alpaugh R. K., Marks J. D. and Weiner L. M. High affinity restricts the localization and tumour penetration of single-chain Fv antibody molecules. Cancer Res, 61, 4750-4755 (2001).
Bird R. E., Hardman K. D., Jacobson J. W., Kaufman B. M., Lee S. M., Lee T., Pope S. H., Riordan G. S. and Whitlow M., Single-chain antigen-binding proteins. Science, 241, 423-426 (1988).
Davies J. and Riechmann L., Camelizing human antibody fragments: NMR studies on VH domains. FEBS Lett, 339, 285-290 (1994)
de Haard H. J., van Neer N., Reurs A., Hufton S. E., Roovers R. C., Henderikx P., de Bruine A. P., Arends J. W. and Hoogenboom H. R., A large non-immunized human Fab fragment phage library that permits rapid isolation and kinetic analysis of high affinity antibodies. J Biol Chem, 274, 18218-18230 (1999)
De Nardo, G. L., Kroger, L. A., Mirick, G. R., Lamborn, K. R. and De Nardo, S. J, Analysis of antiglobulin (HAMA) response in a group of patients with B-lymphocytic malignancies treated with ¹³¹I-Lym-1. Int. J. Biol. Markers, 10 (2), 67-74 (1995)
Farah R. A., Clinchy B., Herrera L. and Vitetta E. S., The development of monoclonal antibodies for the therapy of cancer. Crit Rev Eukaryot Gene Expr, 8, 321-345 (1998)
Frenken L., van der Linden R. H. J., Hermans P. W. J. J., Bos W., Ruuls R. C., de Geus B. and Verrips T., Isolation of antigen-specific llama V_{H}H antibody fragment and their high level secretion by Saccharomyces cerevisiae. J Biotechnol, 78, 11-21 (2000)
Fujimori K., Covell D. G., Fletcher J. E. and Weinstein J. N., A modeling analysis of monoclonal antibody percolation through tumours: a binding site barrier. J Nucl Med, 31, 1191-1198 (1990)
Ghahroudi M. A., Desmyter A., Wyns L., Hamers R. and Muyldermans S., Selection and identification of single domain antibody fragments from camel heavy-chain antibodies. FEBS Letters, 414, 521-526 (1997)
Griffiths A. D., Williams S. C., Hartley O., Tomlinson I. M., Waterhouse P., Crosby W. L., Kontermann R. E., Jones P. T., Low N. M., Allison T. J., et al., Isolation of high affinity human antibodies directly from large synthetic repertoires. EMBO J, 13, 3245-3260 (1994)
Gruber R., van Haarlem L. J., Warnaar S. O., Holz E. and Riethmuller G., The human antimouse immunoglobulin response and the anti-idiotypic network have no influence on clinical outcome in patients with minimal residual colorectal cancer treated with monoclonal antibody CO17-1A. Cancer Res, 60 (7), 1921-1926 (2000)
Hamers-Casterman C., Atarhouch T., Muyldermans S., Robinson G., Hamers C., Bajyana Songa E., Bendahman N. and Hamers R., Naturally occurring antibodies devoid of light chains. Nature, 363, 446-448 (1993)
Hudson P. J., Recombinant antibody fragments. Curr Opin Biotechnol, 9, 395-402 (1998)
Muyldermans S. and Lauwereys M., Unique single-domain antigen binding fragments derived from naturally occurring camel heavy-chain antibodies. J Mol Recognit, 12, 131-140 (1999)
Muyldermans S., Atarhouch T., Saldanha J., Barbosa J. A. R. G. and Hamers R., Sequence and structure of VH domain from naturally occurring camel heavy chain immunoglobulins lacking light chains. Protein Eng, 7, 1129-1135 (1994)
Padlan E. A., Anatomy of the antibody molecule. Mol. Immunol., 31, 169-217 (1994).
Remels L. and De Baetselier P., Characterization of 3LL-Tumour variants generated by in vitro macrophage-mediated selection. Int J Cancer, 39, 343-352 (1987)
Renner C., Hartmann F, Jung W., Deisting C., Juwana M and Pfreundschuhe M., Initiation of humoral and cellular immune responses in patients with refractory Hodgkin's disease by treatment with an anti-CD16 bispecific antibody. Cancer Immunol. Immunother, 49 (3), 173-180 (2000)
Riechmann L. and Muyldermans S., Single domain antibodies: comparison of camel VH and camelised human VH domains. J Immunol Methods, 231, 25-38 (1999)
Schier R., McCall A., Adams G. P., Marshall K., Yim M., Merritt H., Crawford R. S., Weiner L. M., Marks C. and Marks J. D., Isolation of picomolar affinity anti-c-erB2 single-chain Fv by molecular evolution of the complementarity determining regions in the centre of the antibody combining site. J Mol Biol, 263, 551-567 (1996)
Svensson H. P., Frank I. S., Berry K. K. and Senter P., Therapeutic effects of monoclonal antibody-β-lactamase conjugates in combination with a nitrogen mustard anticancer prodrug in models of human renal cell carcinoma. J. Med. Chem., 41, 1507-1512 (1998)
Vanden Driessche T., Verschueren H., Verhaegen S., Van Hecke D. and De Baetselier P., Experimental analysis of the metastatic phenotype of malignant leukocytes. Anti-Cancer Res, 11, 4-73 (1991)
Vaughan T. J., Williams A. J., Pritchard K., Osbourn J. K., Pope A. R., Earnshaw J. C., McCafferty J., Hodits R. A., Wilton J. and Johnson K. S., Human antibodies with sub-nanomolar affinities isolated from a large non-immunized phage display library. Nat Biotechnol, 14, 309-314 (1996)
Viti F., Tarli L., Giovannoni L., Zardi L. and Neri D., Increased binding affinity and valence of recombinant antibody fragments lead to improved targeting of tumoural angiogenesis. Cancer Res, 59, 347-352 (1999)
Vu K. B., Ghahroudi M. A., Wyns L. and Muyldermans S., Comparison of llama VH sequences from conventional and heavy-chain antibodies. Mol Immunol, 34, 1121-1131 (1997)
Ward E. S., Güssow D., Griffiths A. D., Jones P. T. and Winter G., Binding activities of a repertoire of single immunoglobulin variable domains secreted from E. coli. Nature, 341, 544-546 (1989)
Whitlow M., Bell B. A., Feng S. L., Filpula D., Hardman K. D., Hubert S. L., Rollence M. L., Wood J. F., Schott M. E., Milenic D. E., Yokota T. and Schlom J., An improved linker for scFv with reduced aggregation and enhanced proteolytic stability. Protein Eng, 6, 989-993 (1993)
Zeng Z. C., Tang Z. Y., Liu K. D., Lu J. Z., Cai X. J. and Xie H., Human anti-(murine Ig) antibody responses in patients with hepatocellular carcinoma receiving intrahepatic arterial ¹³¹I-labeled Hepama-1 mAb. Preliminary results and discussion. Cancer Immunol. Immunother, 39 (5), 332-336 (1994).

1. An immunoconjugate, devoid of a light chain, which immunoconjugate specifically binds to a tumor antigen, said immunoconjugate comprising: at least one variable domain of a heavy chain antibody having an anti-tumor agent attached thereto, wherein said immunoconjugate inhibits the growth of tumor cells expressing said tumor antigen, leading to a reduction in tumor mass.

2. The immunoconjugate of claim 1, wherein said reduction of tumor mass is at least by 50%.

3. The immunoconjugate of claim 1, which specifically binds to carcinoembryonic antigen (CEA).

4. The immunoconjugate of claim 2, which specifically binds to carcinoembryonic antigen (CEA).

5. The immunoconjugate of claim 1, wherein said variable domain of a heavy chain antibody is a single-domain heavy chain antibody derived from a camelid.

6. The immunoconjugate of claim 2, wherein said variable domain of a heavy chain antibody is a single-domain heavy chain antibody derived from a camelid.

7. The immunoconjugate of claim 3, wherein said variable domain of a heavy chain antibody is a single-domain heavy chain antibody derived from a camelid.

8. The immunoconjugate of claim 4, wherein said variable domain of a heavy chain antibody is a single-domain heavy chain antibody derived from a camelid.

9. The immunoconjugate of claim 1, wherein said anti-tumor agent is an enzyme that activates a prodrug.

10. The immunoconjugate of claim 9, wherein said enzyme is a beta-lactamase.

11. The immunoconjugate of claim 2, wherein said anti-tumor agent is an enzyme that activates a prodrug.

12. The immunoconjugate of claim 11, wherein said enzyme is a beta-lactamase.

13. The immunoconjugate of claim 3, wherein said anti-tumor agent is an enzyme that activates a prodrug.

14. The immunoconjugate of claim 13, wherein said enzyme is a beta-lactamase.

15. The immunoconjugate of claim 4, wherein said anti-tumor agent is an enzyme that activates a prodrug.

16. The immunoconjugate of claim 15, wherein said enzyme is a beta-lactamase.

17. The immunoconjugate of claim 5, wherein said anti-tumor agent is an enzyme that activates a prodrug.

18. The immunoconjugate of claim 17, wherein said enzyme is a beta-lactamase.

19. The immunoconjugate of claim 6, wherein said anti-tumor agent is an enzyme that activates a prodrug.

20. The immunoconjugate of claim 19, wherein said enzyme is a beta-lactamase.

21. The immunoconjugate of claim 7, wherein said anti-tumor agent is an enzyme that activates a prodrug.

22. The immunoconjugate of claim 21, wherein said enzyme is a beta-lactamase.

23. The immunoconjugate of claim 8, wherein said anti-tumor agent is an enzyme that activates a prodrug.

24. The immunoconjugate of claim 23, wherein said enzyme is a beta-lactamase.

25. An immunoconjugate having the polypeptide sequence set forth in SEQ ID NO: 14.

26. A composition comprising the immunoconjugate of any one of claims 1-25 together with an excipient or vehicle.
